(19) United States
(12) Patent Application Publication
(10) Pub. No.: US 2025/0329420 A1
(43) Pub. Date: Oct. 23, 2025

(54) SYSTEM AND METHOD FOR METABOLITE OPTIMIZATION THROUGH GENOME-SCALE COMPARATIVE INTERACTIVE FLUX-DRIVEN REACTION ANALYSIS

(71) Applicant: Yokogawa Electric Corporation, Tokyo (JP)

(72) Inventors: Sunaina BANERJEE, Bengaluru (IN); Rajesh SATHYAMOORTHY, Bengaluru (IN); Vaseef RIZVI, Bengaluru (IN); Maharnob SARKAR, Bengaluru (IN)

(21) Appl. No.: 18/643,911

(22) Filed: Apr. 23, 2024

(51) Int. Cl. G16C 20/10; G16B 5/00; G16B 50/30

(52) U.S. Cl. CPC G16C 20/10; G16B 5/00; G16B 50/30

(57) ABSTRACT

A method, device, and system are disclosed. One example of a method includes loading a model associated with a first organism, where the model expresses a plurality of reaction pathways including one or more intermediate metabolites to produce a target substance from a consumed substance in the first organism, and where the model further provides an association between a first reaction and a second reaction present in each reaction pathway and one or more genes that are used to produce the target substance. The method further includes obtaining a cell design objective function and optimizing one or both of the target substance and the consumed substance using the second flux flow.

### FIELD OF THE DISCLOSURE

The present disclosure generally relates to simulation systems and, more specifically, relates to simulation systems that enable comparative interactive genome-scale flux balance analysis (FBA).

### BACKGROUND

Bio-foundry industries are developed on organisms that produce high value products with high yield. The existing potential of an organism to produce bioactive compounds, industrial precursor compounds, and green alternatives needs to be analyzed for a successful bio-industry. The organism potential and application scope can be expanded further by genetic engineering and optimizing growth parameters which will allow scaling up production in large batches.

There are two major challenges in this field. Currently there are no benchmarking approaches to compare organisms for their metabolic potential (no quantitative methods exist as per our knowledge). Furthermore, every subject matter expert requires a different ‘cell-design objective function’ for optimization based on their target compound. This must be tailormade for the application—for example, obtaining the best yield while not affecting survival of organism, balancing product yield with growth rate, introducing minimal engineering in the organism etc. The cell-design objective function is important to the success of scaling-up organism growth and optimizing the yield of target compounds. However, the cell-design objective function to improve a strain is complex and cannot be guessed a priori.

### SUMMARY

Genome-scale metabolic models (GSMM) are unique for each strain and organism. The GSMM is a complex structure, which captures information about all the metabolites present in an organism, reactions that occur in the organism, and genes that are associated with reactions. A well-curated GSMM and experimentally-derived parameters can be used as an input to perform simulations using various algorithms to optimize the yield of a target compound. This is an iterative process performed until the appropriate objective is derived. An SME thus has to perform multiple iterative simulations. If the simulation output lacks interpretability and the iterations lack automation, the decision-making process presents major deterrents. Thus, a manual search for the right cell-design objective function does not guarantee the quality of results. Further, a lack of interpretability in simulation outputs makes it hard to compare between a pool of organisms due to the complexity of GSMM and simulation results.

These and other needs are addressed by the various embodiments and configurations of the present disclosure. The present disclosure can provide a number of advantages depending on the particular configuration.

Cell factory design broadly refers to choosing an organism, engineering an organism, and enabling its growth under optimal conditions to act as a ‘factory’ for target compound production. Embodiments of the present disclosure propose an interactive human-computer interface which will address the formulation of an appropriate objective function for cell-factory design. Embodiments disclosed herein enable the SME to understand the mathematical output from the GSMM simulation system through representation.

Embodiments of the present disclosure also describe the construction of the Flux-derived Demand-Supply Exchange of Metabolites (FDSeM) and Reaction interaction Graph (RiG), which will enable the SME to: a) Select the right organism that produces greater yield of target compound; b) Select the organism with minimal impurities for downstream process; and c) Improve yields of target compound in organisms while optimizing growth. The GSMM and experimentally derived parameters are used for simulation (Flux balance/flux variability analysis). The FDSeM may include an interactive representation which captures a weighted flux distribution from source metabolite to target metabolite. Additionally, the FDSeM can be used to analyze the metabolite productivity in an organism. A metabolite production fingerprint for an organism can be constructed by interacting with FDSeM and imposing constraints on metabolite ordering based on the attribute (e.g., pathway in which metabolites participate/their chemical properties, etc), followed by bi-clustering.

The RiG can be built from a sequential reaction topology with weighted flux. The RiG can be supported by a gene-reaction database, hence interactively altering a gene expression parameter (e.g., overexpression, deletion, etc.) would allow the SME to tune the production of target compound.

The FDSeM and RiG can be constructed for organisms where a curated GSMM exist. The alignment of FDSeM/RiG across organisms involves non-trivial optimization, where reaction equivalence is established and where reactions are be sorted by pathway/attributes to allow interpretability.

Embodiments of the present disclosure also provide mechanisms to enable the quantitative comparison by involving matrix bi-clustering to allow the comparison between organisms.

Aspects of the present disclosure include linking reactions in RiG to attributes such as gene-protein rule, pathway, reaction features (e.g., growth, survival, toxicity, etc.) with a database. Aspects of the present disclosure also include the ability to impose constraints interactively using the database and ordering reactions.

If there is a major contaminant with a similar chemical profile as a compound-of-interest, the SME can use the simulations with genetic modification and RiG to mine out favorable and minimal gene deletions that will ensure better yield of compound-of-interest, minus impurities. One approach for minimizing contaminants and ensuring the ease of extraction for a compound-of interest may include: fetching reactions associated with the contaminant using in-house database; interactively deleting the genes associated with the reactions (e.g., using gene-protein association database); and computing the RiG for each deletion. Iterative deletions may be performed to: minimize yield of contaminant; optimize yield of compound-or-interest; optimize the biomass objective function; and/or minimize toxicity.

In some aspects, the techniques described herein relate to a method, including: loading a model associated with a first organism, where the model expresses a plurality of reaction pathways including one or more intermediate metabolites to produce a target substance from a consumed substance in the first organism, and where the model further provides an association between a first reaction and a second reaction present in each reaction pathway and one or more genes that are used to produce the target substance; obtaining a cell design objective function that includes one or more constraints on production of the target substance that are useable to optimize the production of the target substance; determining a first flux flow between the first reaction and the second reaction, where the first reaction and the second reaction are performed to produce the target substance using the consumed substance; modifying the model by changing at least one of the association between the first reaction and the second reaction to yield a modified association and by changing the one or more genes that are used to produce the target substance, thereby providing a modified model; determining a second flux flow between the first reaction and the second reaction using the modified model; and optimizing one or both of the target substance and the consumed substance using the second flux flow.

In some aspects, the techniques described herein relate to a system, including: a processor; and a memory device coupled with the processor, where the memory device includes data stored thereon that, when processed by the processor, enables the processor to: load a model associated with a first organism, where the model expresses a plurality of reaction pathways including one or more intermediate metabolites to produce a target substance from a consumed substance in the first organism, and where the model further provides an association between a first reaction and a second reaction present in each reaction pathway and one or more genes that are used to produce the target substance; obtain a cell design objective function that includes one or more constraints on production of the target substance that are useable to optimize the production of the target substance; determine a first flux flow between the first reaction and the second reaction, where the first reaction and the second reaction are performed to produce the target substance using the consumed substance; modify the model by changing at least one of the association between the first reaction and the second reaction to yield a modified association and by changing the one or more genes that are used to produce the target substance, thereby providing a modified model; determine a second flux flow between the first reaction and the second reaction using the modified model; and optimize one or both of the target substance and the consumed substance using the second flux flow.

In some aspects, the techniques described herein relate to a non-transitory computer-readable medium having processor-executable instructions stored thereon, where the instructions enable a processor, when executed, to: load a model associated with a first organism, where the model expresses a plurality of reaction pathways comprising one or more intermediate metabolites to produce a target substance from a consumed substance in the first organism, and where the model further provides an association between a first reaction and a second reaction present in each reaction pathway and one or more genes that are used to produce the target substance; obtain a cell design objective function that includes one or more constraints on production of the target substance that are useable to optimize the production of the target substance; determine a first flux flow between the first reaction and the second reaction, where the first reaction and the second reaction are performed to produce the target substance using the consumed substance; modify the model by changing at least one of the association between the first reaction and the second reaction to yield a modified association and by changing the one or more genes that are used to produce the target substance, thereby providing a modified model; determine a second flux flow between the first reaction and the second reaction using the modified model; and optimize one or both of the target substance and the consumed substance using the second flux flow.

Aspects of the present disclosure also contemplate one or more means for performing any one or more of the above aspects or aspects of the embodiments described herein.

The preceding is a simplified summary of the invention to provide an understanding of some aspects of the invention. This summary is neither an extensive nor exhaustive overview of the invention and its various embodiments. It is intended neither to identify key or critical elements of the invention nor to delineate the scope of the invention but to present selected concepts of the invention in a simplified form as an introduction to the more detailed description presented below. As will be appreciated, other embodiments of the invention are possible utilizing, alone or in combination, one or more of the features set forth above or described in detail below. Also, while the disclosure is presented in terms of exemplary embodiments, it should be appreciated that an individual aspect of the disclosure can be separately claimed.

### BRIEF DESCRIPTION OF THE DRAWINGS

The present disclosure is described in conjunction with the appended figures:

FIG. 1 depicts a computing system in accordance with embodiments of the present disclosure;

FIG. 2 depicts a simulation pipeline in accordance with embodiments of the present disclosure;

FIG. 3 depicts a processing for making simulation data interpretable for cell factory design in accordance with embodiments of the present disclosure;

FIG. 4 depicts one example of a simulation process in accordance with embodiments of the present disclosure;

FIG. 5 depicts a simulation process employing flux-derived demand-supply exchange of metabolites (FDSeM) in accordance with embodiments of the present disclosure;

FIG. 6 depicts a simulation process employing a reaction interface graph (RiG) in accordance with embodiments of the present disclosure;

FIG. 7 depicts a process to quantitate the production of metabolite(s) in an organism in accordance with embodiments of the present disclosure;

FIG. 8 depicts another example of a simulation process in accordance with embodiments of the present disclosure;

FIG. 9 depicts another example of a simulation process in accordance with embodiments of the present disclosure;

FIG. 10 depicts another example of a simulation process in accordance with embodiments of the present disclosure; and

FIG. 11 depicts another example of a simulation process in accordance with embodiments of the present disclosure.

### DETAILED DESCRIPTION

The ensuing description provides embodiments only and is not intended to limit the scope, applicability, or configuration of the claims. Rather, the ensuing description will provide those skilled in the art with an enabling description for implementing the embodiments. It will be understood that various changes may be made in the function and arrangement of elements without departing from the spirit and scope of the appended claims.

Any reference in the description comprising a numeric reference number, without an alphabetic sub-reference identifier when a sub-reference identifier exists in the figures, when used in the plural, is a reference to any two or more elements with the like reference number. When such a reference is made in the singular form, but without identification of the sub-reference identifier, it is a reference to one of the like numbered elements, but without limitation as to the particular one of the elements being referenced. Any explicit usage herein to the contrary or providing further qualification or identification shall take precedence.

Before describing the various embodiments, it is helpful to understand the possible meaning of various terms used herein.

An “organism” as used herein may refer to a cluster of cells. An organism may include one or more attributes defined by a genome.

A “cell” as used herein may refer to a cluster of pathways. A cell may include one or more attributes defined by a genome.

A “pathway” as used herein may refer to a cluster of reactions. A pathway may include one or more attributes defined by its role in a cell (e.g., survival, carbon, metabolism, etc.).

A “reaction subsystem” as used herein may refer to a niche cluster of reactions.

A “reaction” as used herein may refer to a cluster of metabolites (e.g., input and/or output). Oxidation, proton transfer, and metabolite transport are some example classes of a reaction. An enzyme driving a reaction may be linked to the reaction.

An “enzyme” or “protein” as used herein may refer to a product of a gene. Attributes of an enzyme or protein may include annotations on cellular location, structure, whether a transporter, whether an enzyme, or whether part of a complex, etc.

A “gene” as used herein may refer to a functional segment of a genome. An attribute of a gene may include a gene class.

A “nutrient” as used herein may refer to an element that is fed to a cell. An attribute of a “nutrient” may include a growth media with component quantitated.

A “metabolite” as used herein may refer to an output or product of nutrients and internal reactions. An attribute of a metabolite may include internal molecules of a cell as well as chemical attributes like polarity, hydrophobicity, and/or toxicity.

A “target compound” or “target substance” as used herein may refer to an element that is extracted from a reaction. A target compound or target substance may correspond to waste to the cell (e.g., a by-product), but may be considered valuable to industry.

The term “flux” as used herein may refer to the rate or rates to alter or optimize.

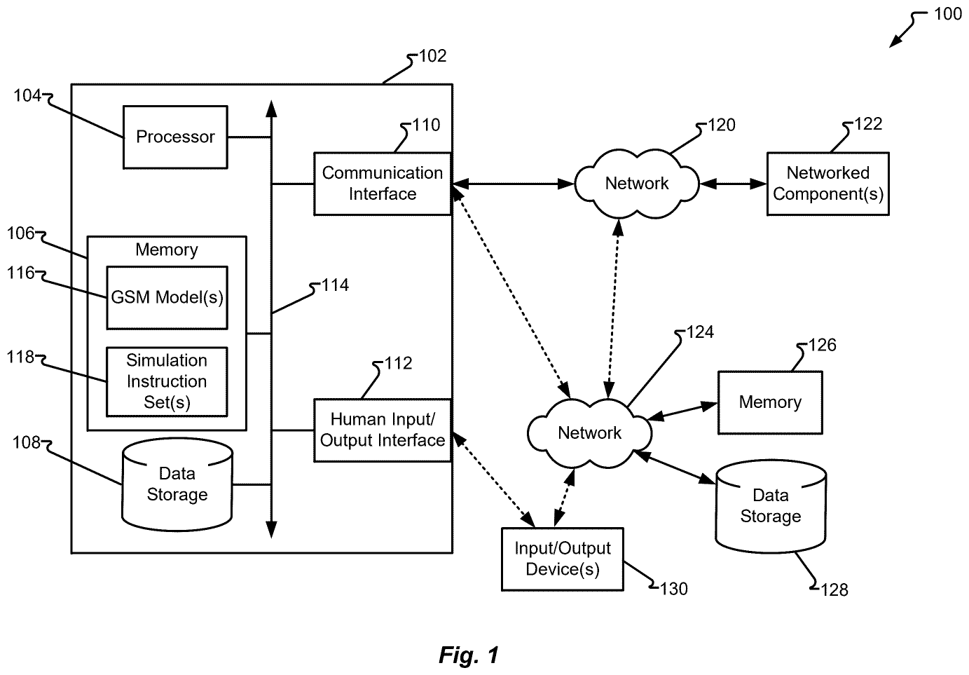

FIG. 1 depicts an example computing system 100 in accordance with embodiments of the present disclosure. As will be discussed in further detail herein, the computing system 100 may be utilized to implement one or more simulations that facilitate decisions on cell factory design and/or optimization.

In one embodiment, the computing system 100 may include a computing device 102 comprising various components and connections to other components and/or computing devices to execute certain embodiments described herein. The components of the computing device 102 are variously embodied and may comprise a processor 104 and memory 106. The term “processor,” as used herein, may refer to any suitable type of processing device and/or micro processing device. Illustratively, but without limitation, the processor 104 may include an Integrated Circuit (IC) chip, a microprocessor, a Central Processing Unit (CPU), and Graphics Processing Unit (GPU), an Application Specific Integrated Circuit (ASIC), a Field-Programmable Gate Array (FPGA), a semiconductor device, combinations thereof, and the like.

Processor 104 may include programmable logic functionality, such as determined, at least in part, from accessing machine-readable instructions maintained in a non-transitory data storage, which may be embodied as circuitry, on-chip read-only memory, computer memory 106, data storage 108, etc., that cause the processor 104 to perform the steps or processes according to the instructions. Processor 104 may be embodied as a single electronic microprocessor or multiprocessor device (e.g., multicore) having electrical circuitry therein which may further comprise a control unit(s), input/output unit(s), arithmetic logic unit(s), register(s), primary memory, and/or other components that access information (e.g., data, instructions, etc.), such as received via bus 114, executes instructions, and outputs data, again such as via bus 114.

In some embodiments, processor 104 may include a shared processing device that may be utilized by other processes and/or process owners, such as in a processing array within a system (e.g., blade, multi-processor board, etc.) or distributed processing system (e.g., “cloud”, farm, etc.). It should be appreciated that processor 104 is a non-transitory computing device (e.g., electronic machine comprising circuitry and connections to communicate with other components and devices). Processor 104 may operate a virtual processor, such as to process machine instructions not native to the processor (e.g., translate the VAX operating system and VAX machine instruction code set into Intel® 9xx chipset code to enable VAX-specific applications to execute on a virtual VAX processor). However, as those of ordinary skill understand, such virtual processors are applications executed by hardware, more specifically, the underlying electrical circuitry and other hardware of the processor (e.g., processor 104). Processor 104 may, alternatively or additionally, be executed by virtual processors, such as when applications (i.e., Pod) are orchestrated by Kubernetes. Virtual processors enable an application to be presented with what appears to be a static and/or dedicated processor executing the instructions of the application, while underlying non-virtual processor(s) are executing the instructions and may be dynamic and/or split among a number of processors.

In addition to the components of processor 104, computing device 102 may utilize computer memory 106 and/or data storage 108 for the storage of accessible data, such as instructions, values, etc. Examples of data and/or instructions that may be stored in memory 106 include, without limitation, one or more GSM models 116 and one or more simulation instruction sets 118. As will be described in further detail herein, the processor 104 may be configured to access the simulation instruction set(s) 118, which load GSM model(s) 116 as part of implementing a simulation or multiple simulations to support an analysis of target substance production from an organism given a set of constraints.

The computing device 102 may further include a communication interface 110 that facilitates communication with other devices in the system 100. As illustrated, the communication interface 110 may provide connectivity between the computing device 102 and a communication network 120, 124. The communication network(s) 120, 124 may provide machine-to-machine connectivity and communication, thereby enabling a user to remotely access components of the computing device 102. Communication interface 110 may be embodied as a network port, card, cable, or other configured hardware device.

Additionally or alternatively, human input/output interface 112 connects to one or more interface components to receive and/or present information (e.g., instructions, data, values, etc.) to and/or from a human and/or electronic device. Examples of input/output devices 130 that may be connected to input/output interface include, but are not limited to, keyboard, mouse, trackball, printers, displays, sensor, switch, relay, speaker, microphone, still and/or video camera, etc. In another embodiment, communication interface 110 may comprise, or be comprised by, human input/output interface 112. Communication interface 110 may be configured to communicate directly with a networked component or configured to utilize one or more networks, such as network 120 and/or network 124.

Network 120 may include a wired network (e.g., Ethernet), wireless (e.g., WiFi, Bluetooth, cellular, etc.) network, or combination thereof and enable computing device 102 to communicate with networked component(s) 122. In other embodiments, network 120 may be embodied, in whole or in part, as a telephony network (e.g., public switched telephone network (PSTN), private branch exchange (PBX), cellular telephony network, etc.).

Additionally or alternatively, one or more other networks may be utilized. For example, network 124 may represent a second network, which may facilitate communication with components utilized by computing device 102. For example, network 124 may be an internal network to a business entity or other organization, whereby components are trusted (or at least more so) than networked components 122, which may be connected to network 120 comprising a public network (e.g., Internet) that may not be as trusted.

Components attached to network 124 may include computer memory 126, data storage 128, input/output device(s) 130, and/or other components that may be accessible to processor 104. For example, computer memory 126 and/or data storage 128 may supplement or supplant computer memory 106 and/or data storage 108 entirely or for a particular task or purpose. As another example, computer memory 126 and/or data storage 128 may be an external data repository (e.g., server farm, array, “cloud,” etc.) and enable computing device 102, and/or other devices, to access data thereon. Similarly, input/output device(s) 130 may be accessed by processor 104 via human input/output interface 112 and/or via communication interface 110 either directly, via network 124, via network 120 alone (not shown), or via networks 120 and 124. Each of computer memory 106, data storage 108, computer memory 126, data storage 128 may comprise a non-transitory data storage comprising a data storage device.

It should be appreciated that computer readable data may be sent, received, stored, processed, and presented by a variety of components. It should also be appreciated that components illustrated may control other components, whether illustrated herein or otherwise. For example, one input/output device 130 may be a router, a switch, a port, or other communication component such that a particular output of processor 104 enables (or disables) input/output device 130, which may be associated with network 120 and/or network 124, to allow (or disallow) communications between two or more nodes on network 120 and/or network 124. One of ordinary skill in the art will appreciate that other communication equipment may be utilized, in addition or as an alternative, to those described herein without departing from the scope of the embodiments.

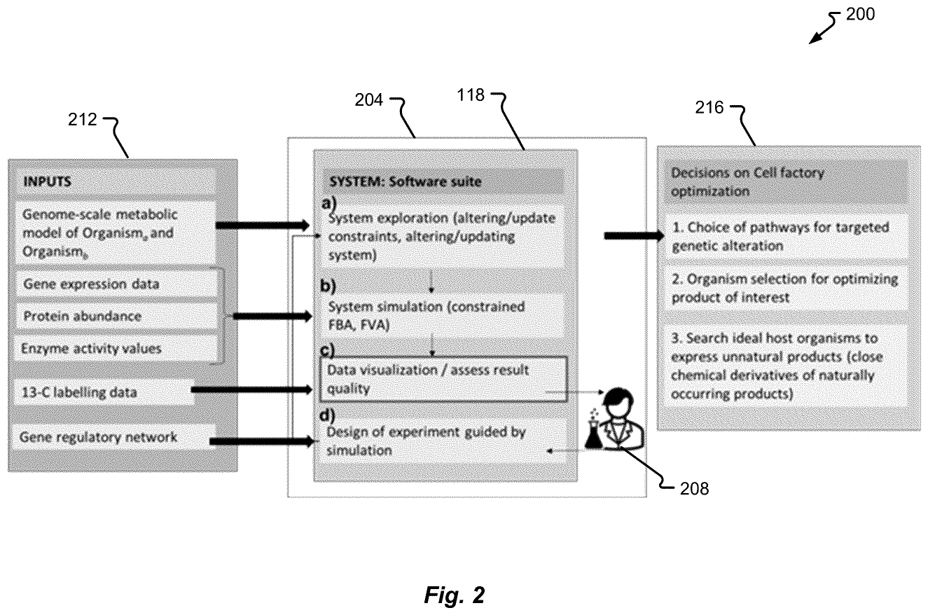

Referring now to FIG. 2, additional details of a simulation pipeline 200 will be described in accordance with at least some embodiments of the present disclosure. The simulation pipeline 200 may be implemented, entirely or in part, by the processor 104 executing the simulation instruction set(s) 118 stored in memory 106. In some embodiments, the simulation instruction set(s) 118 may be included as part of a simulation software package 204 that receives one or more inputs 212 and produces one or more outputs 216 based on a processing of the one or more inputs 212. In some embodiments, a graphical user interface (GUI) for the simulation software package 204 may be presented via a display device, such as an input/output device 130. Presentation of the GUI via the display device may enable a user 208 to define one or more inputs 212 for the simulation, define one or more constraints to impose on the simulation, and/or view one or more outputs 216 generated by the simulation software package 204.

In accordance with at least some embodiments, the simulation software package 204 provides the capability for a human-computer interaction. By interacting with the simulation software package 204, the user 208 can execute the simulation pipeline 200 to address the formulation of a complex ‘objective function’ for organism optimization and cell factory design. The simulation software package 204 is illustrated to include a) an exploration module to interactively load the genome-scale metabolic model and input initial constraints b) a simulation module to execute a simulation with a desired starting objective (target compound production) c) a data visualization module to make the simulation data interpretable and/or visible to the user 208 based on the objective function and d) an iteration module where further simulation(s) can be specified for design of experiment.

The simulation software package 204 may be configured to receive a number of different inputs to support the simulation process(es) as disclosed herein. Examples of inputs 212 that may be provided to the simulation software package 204 include, without limitation, GSM model(s) 116, gene expression data, protein abundance data, enzyme activity values, 13-C labelling data, and gene regulatory network data.

Based on the processing of inputs 212, the simulation software package 204 may generate one or more outputs 216. The outputs 216 may be provided to another computing device for further processing and/or may be presented to the user 208 via the display device(s) as described herein. Examples of outputs 216 that may be provided by the simulation software package 204 include, without limitation, pathway choices for targeted genetic alteration, organism selection for optimizing a product of interest, and/or searching ideal host organism(s) to express unnatural products.

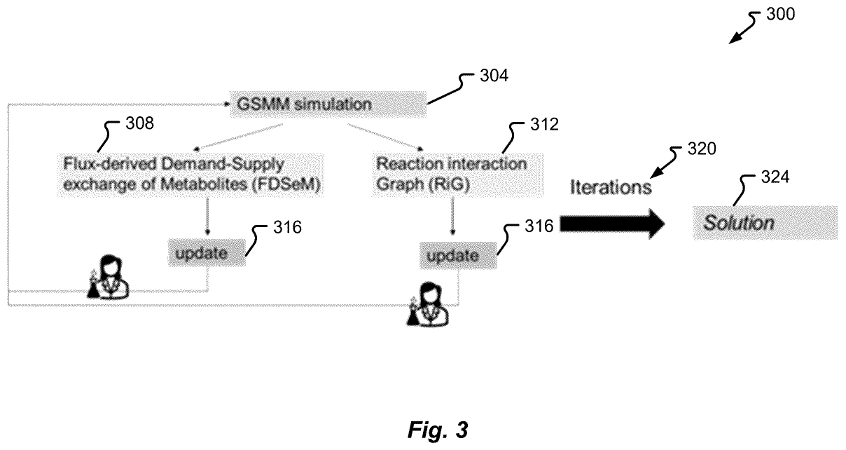

As can be seen in FIG. 3, a process 300 for making simulation data interpretable for cell factory design is illustrated. The process 300 may include performing the GSMM simulation 304 (e.g., with the simulation software package 204). The GSMM simulation 304 may produce one or more outputs 216, which include a presentation of an FDSeM 308 and/or a presentation of a RiG 312. A user 208 may be enabled to update 316 the GSMM simulation 304 through a number of simulation iterations 320. Each iteration 320 may produce a different set of outputs, which eventually lead the user 208 to a solution 324 for the cell factory design.

It should be appreciated that the FDSeM 308 or RiG 312 may produce an output 216 that is eventually adopted by the user 208 as a solution 324. In some embodiments, outputs from multiple simulations, including outputs from the FDSeM 308 and/or RiG 312 may produce the solution 324.

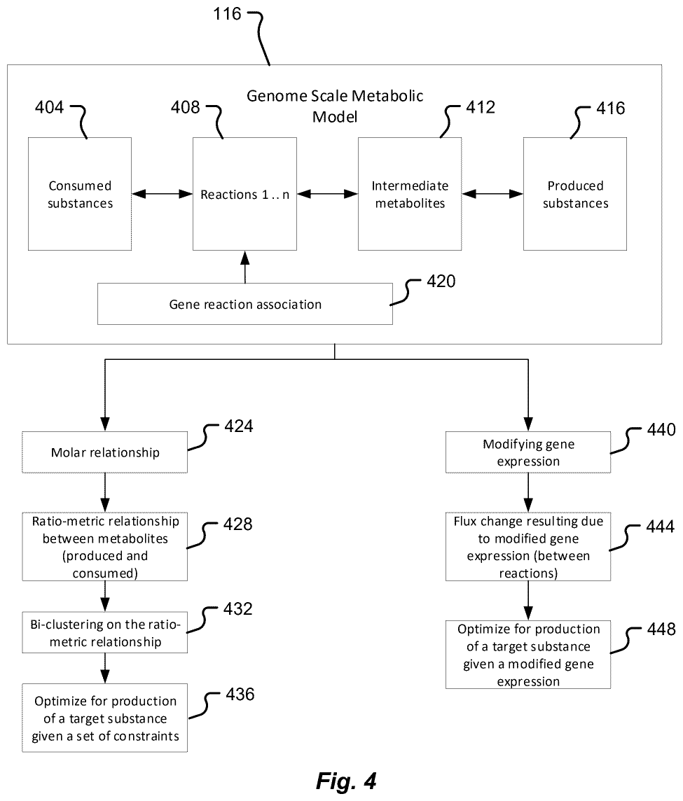

FIG. 4 illustrates additional details of a simulation process that utilizes one or more GSMMs 116. In accordance with at least some embodiments, the GSMM 116 may include data describing consumed substances 404, data describing reaction(s) 408, data describing intermediate metabolites 412, and data describing produced substances 416. The GSMM 116 may also include data describing gene reaction associations 420. In some embodiments, the data from the GSMM 116 may be used to simulate the target substance production and normalize such that comparison is possible. Employing a simulation process as described herein with the GSMM 116 may also help derive a ratio-metric flux relationship between produced and consumed substances, study the impact of modified gene expression on reaction/fluxes resulting in produced substances, and reference bi-clustering of organisms with constraints to maximize metabolite production.

In some embodiments, the simulation process may utilize the GSMM 116 to define a molar relationship 424, define a ratio-metric relationship between metabolites (e.g., produced and/or consumed) 428, perform bi-clustering on the ratio-metric relationship 432, and optimize for production of a target substance given a set of constraints 436. In some embodiments, the simulation process may utilize the GSMM 116 to modify a gene expression 440, determine a flux change due to the modified gene expression 444, and optimize for production of a target substance give a modified gene expression 448. Additional details of both simulation processes are provided hereinbelow.

Simulation leveraging the FDSeM 308 may express weighted fluxes due to the uptake of nutrients in media. In some embodiments, a bipartite reaction network 504 may be generated from the GSMM 116. Additionally, weighted fluxes can be represented in a source-target metabolite matrix 508. The internal fluxes are computed for each reaction in the organism and the FDSeM 308 produces an output 512 that allows an assessment of net depletion of metabolites and net production of metabolites. The net production of metabolites can be analyzed by summing the columns of FDSeM 308.

In some embodiments, an organism can be considered an industrial park. A single cell of the organism can be considered a factory. A single cell of the organism consumes raw materials (nutrients) so that energy is generated to support growth, development, and life processes. A reaction in a cell is connected to some entities viz., input metabolite, enzyme which acts as catalyst for the reaction, output metabolites created at the end of reaction. Reactions are also associated with pathways that allow life processes. Pathways are like sub-divisions of a factory and reactions are clubbed to form processes under the pathways.

Since the cell factory has ordering and hierarchy, a series of steps can be used to understand the working of the factory. In some embodiments, the GSMM 116 captures a partial blueprint of the factory. The GSMM 116 may store data describing: nutrients that are being consumed by the cell, how those nutrients are processed, how energy is being generated, all the processed that allows the cell to survive, the other participants in the cell such as diverse range of metabolites (with distinct chemical natures), the protein enzymes and transporters which use the metabolites/nutrients, and/or the reactions that the metabolites and enzymes participate in.

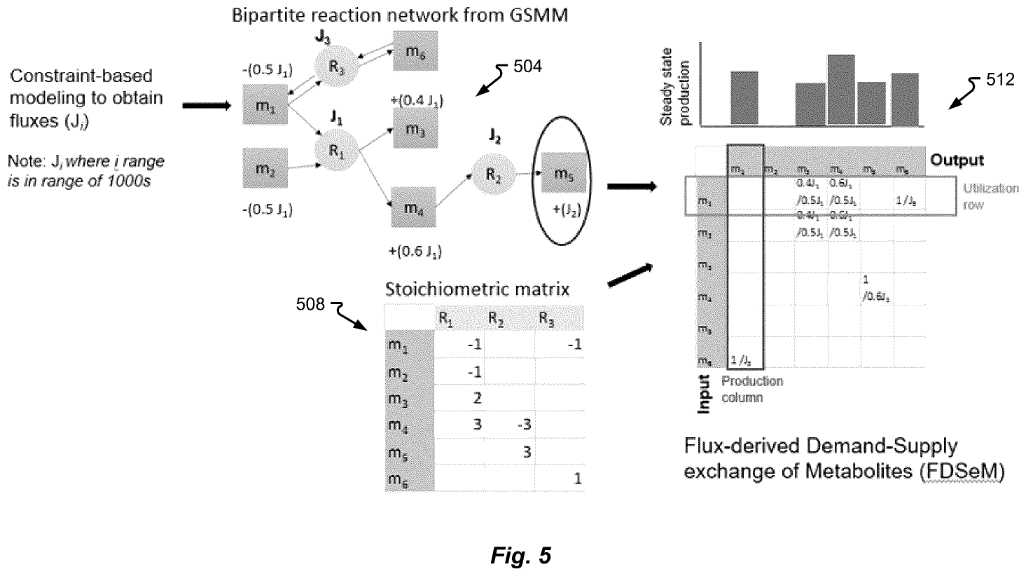

Organizing information about metabolites and enzymes in the analysis provided in FIG. 5 helps the user 208 understand the overall cell factory. In particular, metabolites may be associated to certain pathways as shown in the network 504. Metabolites are also connected to reactions based on their participation in a standard formalism (e.g., the stoichiometric matrix 508). Metabolites can be connected to each other based on if a metabolite acts as raw material to become another metabolite (e.g., act as an input for a reaction). As a non-limiting example, A+B→C+D, which implies A, B can be linked to C and D in a directed manner. This understanding allows the construction of a matrix 512 where input elements are on one side and output elements are ordered on another side.

Another case is: A+B↔C+D, which implies A, B are linked to C and D. The rates driving the formation of C, D may be different than a backward reaction linked to forming A, B. This nuance can also be captured in the stoichiometric matrix 508. The matrix 512 linking metabolites of a cell also captures information about the steady state rates (fluxes) of consumption and formation of metabolites. In some embodiments, each cell of matrix 512 is filled with a weighted contribution of input metabolite flux to output metabolite flux.

Since the output matrix 512 of FDSeM 308 can be ordered with input metabolites on rows and output metabolites on columns, further analysis is possible. Specifically, row values can be analyzed to estimate a sum consumption and column values can be analyzed to estimate sum production. Additionally, the input raw material concentration and the rates at steady state (production and consumption) can be summed to describe the net production for each metabolite. This allows the user 208 to analyze the state of production.

At this stage, the FDSeM 308 captures how raw materials are being utilized after loading onto the cell factory (e.g., cell uptake) to how the raw material makes the product (e.g., the target metabolite of interest) by means of precursors (e.g., intermediate metabolites).

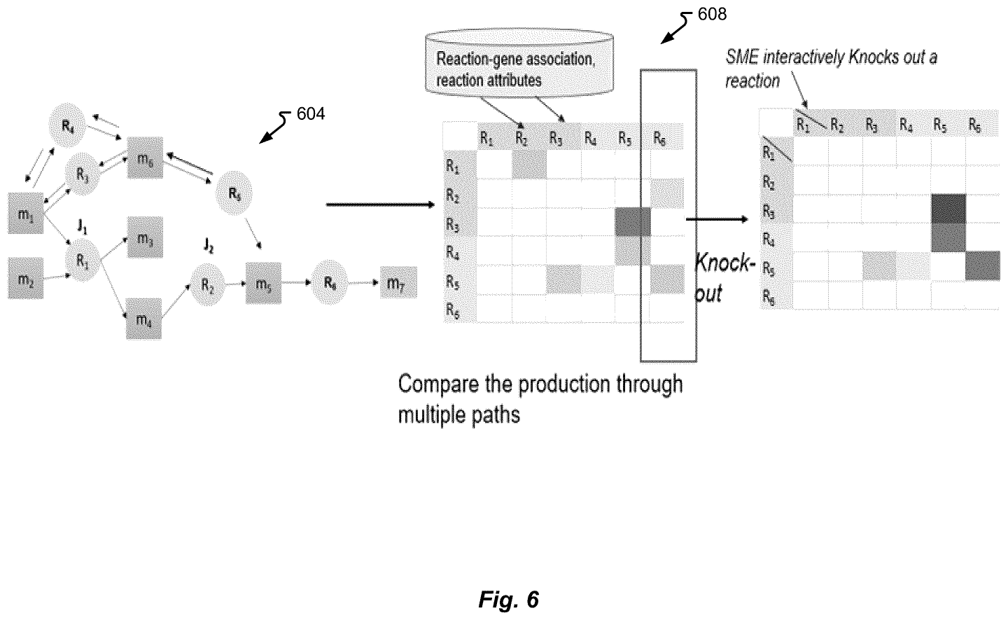

FIG. 6 provides another mechanism for analyzing another layer of the factory blueprint. In particular, it may also be desirable to analyze how processes are connected to one another. As an example, it may be useful to differentiate between processes that are fast and efficient versus processes that are slow. It may also be useful to determine how processes are connected versus which processes are isolated. As can be seen in FIG. 6, reactions (just like metabolites) belong to certain pathways. Many reactions in sequence make up a process, many processes make a pathway. Such information be stored in a GSMM 116 and/or some other data structure stored in data storage 108. The pathway information and reaction information may be organized in a reaction network 604. In the reaction network 604, reactions are connected to each other immediately when they share common participating metabolites (either input or output). With the reaction network 604, a matrix of reactions 608 can be produced by ordering precursor reactions in a row and successive reactions in a column. The steady state rates can be converted to a ratio-metric factor and used to fill the cells of the matrix of reactions 608. The matrix of reactions 608 may also be referred to as the RiG 312 or an output thereof. In the RiG 312 (or matrix of reactions 608), all connected reactions will have a value in the cell, whereas non-connected reactions may provide null cells. Accordingly, the matrix of reactions 608 or RiG 312 may be considered a sparse matrix.

Reactions are processes, and enzymes are the machines that drive processes. Enzymes themselves are produced from genes (higher level of control). Genes are controllers for each machine. In some embodiments, a user 208 can switch off the master controller (gene) and shut down a specific machine (enzyme)—this will disrupt the specific process (reaction). Of course this will affect neighboring processes. A user 208 may also increase the rate of production through a specific machine by amplifying the controller (gene)—this will lead to overproduction of few raw materials and change the flux of production. The RiG 312 interactively allows the user 208 to switch off (delete) or amplify (overexpress) the controllers (genes). This will allow the user 208 to simulate and recalculate process effects arising from either of the modifications described above. In some embodiments, a database may be used to link genes to enzymes at the backend of the RiG 312.

As can be appreciated, a link exists between a source to target reaction when the products of source reaction become inputs of target reaction. Hence the network topology described herein captures the reaction flow of organism. Further the weighted flux populating the RiG 312 provide an estimate of high flow and low flow paths that may lead to a product of interest. The RiG 312 is also associated with gene-protein rules database, where genes that control enzymes for a reaction are stored.

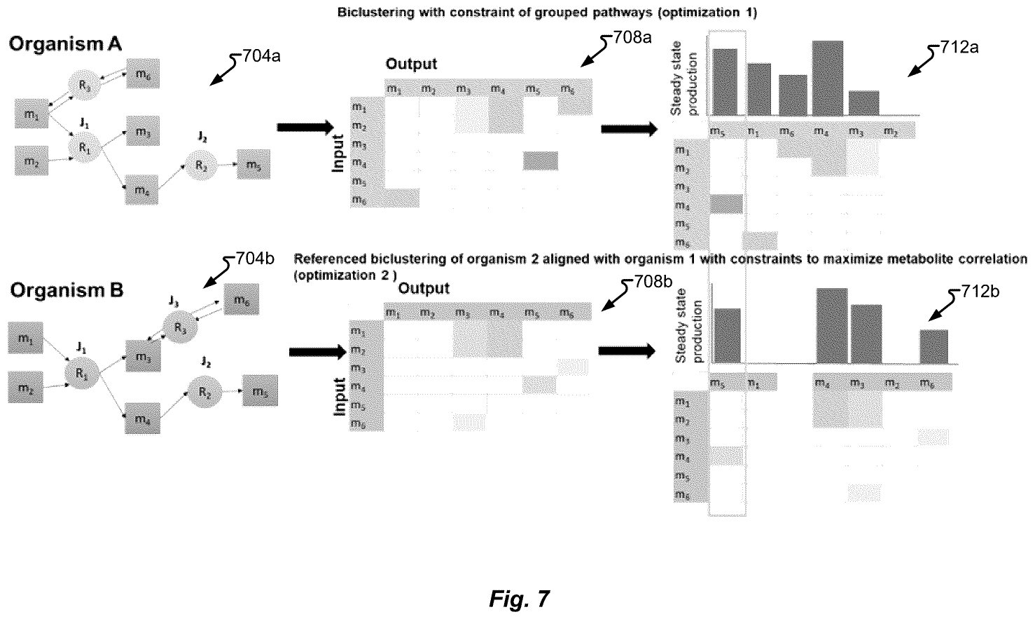

FIGS. 5 and 6 highlight how to use the FDSeM 308 and RiG 312 for simulations in cases where the user 208 desires to optimize the productivity of a single cell. In some embodiments, the FDSeM 308 and RiG 312 can be used to compare the productivity of two different organisms with different genomes and reactions, but where the product of interest is common. Additional details of this approach will be described with reference to FIG. 7, in which unique layouts of processes (reactions, Ri) and raw materials which are being converted to products (metabolites, mi).

In Organism A there is a single pathway in the reaction network 704a to produce the compound of interest ms. Likewise, in Organism B there is also a single pathway in the reaction network 704b to produce the same compound of interest ms. However, in Organism B there are processes that use up the raw materials for some other compound, in addition to making ms. There is a layout difference in the blueprints of each Organism. Sometimes, more than one route may exist to create a product such as ms. Thus, embodiments of the present disclosure contemplate the ability to have a flexible platform to compare the two organisms, their constituent processes and productivities.

We can make decisions by comparing the outputs 708a, 708b of each Organism, as well as the layouts in FDSeM 712a, 712b of each Organism. Here the shunting of raw materials may be represented without directly representing the process. It makes the analysis more direct, less complicated, and easier for user 208 to understand.

To make the comparison interpretable, the following process may be executed:
a) Select one of the organisms being compared as a reference
b) Annotate the raw materials (mi) based on chemical attributes like formula, hydrophobicity, etc. using a database
c) Specify an attribute (process association or chemical property) based on which the raw materials are sorted. This enriches the neighborhood of the matrix so that related mi are localized and separated from unrelated mi
d) Calculate the FDSeM using the recipe for the reference Organism A
e) Impose a bi-clustering algorithm on the FDSeM. This is Optimization 1, in which the attribute of mi and the amount of utilization of mi are used for sorting the raw materials. The raw materials which are highly used in processes get clustered together whereas those used moderately are clustered together. This is a fingerprint for Organism A.
f) Next, the template of Organism A is used to impose an alignment for Organism B (Optimization 2).
g) The raw materials and processes that are common between Organism A and B are aligned by using the database of attributes.
h) An optimization is performed to find the best match to align the FDSeM 712a of Organism A and the FDSeM 712b of Organism B.
i) There are large number of processes and raw materials. Without the database, drawing a correspondence between the constituents of Organisms A and B is hard to compute and interpret. Similarly, the alignment is complex and hence has to be achieved using optimization.
j) The net productivity of all the compounds produced in Organisms A and B can be compared. This profile is a direct fingerprint and interpretable comparison between Organisms A and B. This can right-away allow the selection of an organism.

If there is no clear-cut choice, and also in complex cases, it may be necessary to make further decisions. Some decisions can be enabled post-optimization and alignment of FDSeM. In some embodiments, it may be possible to retrieve and focus the analysis on all the compounds with similar metabolite profiles as target compound. With this approach, it may become possible to assess if Organism A or Organism B has a better ratio of production between target compound and impurities. In some embodiments, it may also be possible to impose a constraint on the amount of raw materials made available to a process. Thus, it becomes possible to simulate and re-compute FDSeMs 712a, 712b for both organisms. Following the iteration, the user 208 can then choose whether to invest in Organism A or Organism B.

According to at least some embodiments, a compound-of-interest may be naturally produced by many organisms. However, the selection of the ideal or preferred organism to scale-up the growth may depend on many factors which are complex to analyze. The native yields of the compound should be considered. Moreover, during downstream extraction there should not be potential contaminants that are produced in equal proportion to the compound of interest (purity factor).

Hence, embodiments of the present disclosure propose a solution encompassing the following for making a choice of organism: (1) alignment between two organisms where constraints can be imposed on metabolite ordering and (2) quantitative analysis of production of each compound in the organisms of interest.

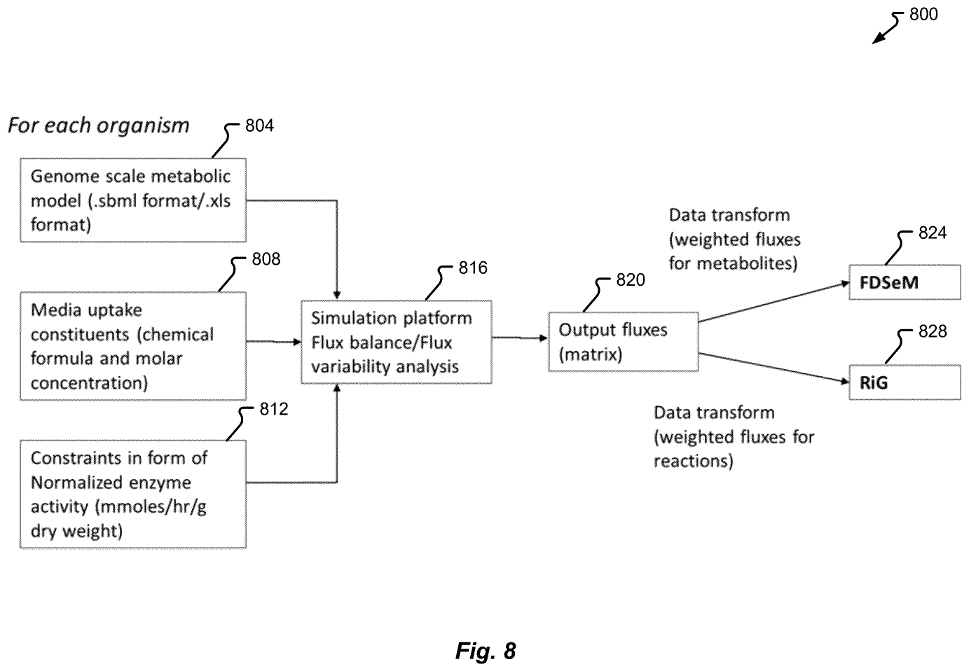

The process to quantitate the production of each metabolite in an organism will now be described with reference to FIG. 8. The inputs for the simulation platform 816 may include a well-curated GSMM 804 of an organism, a description of media uptake constituents, and constraints in the form of normalized enzyme activity 812. In some embodiments, the simulation platform 816 may construct a stoichiometric matrix from the GSMM and experimental data to perform a simulation using the flux balance analysis (FBA) or flux variability analysis (FVA). The output fluxes 820 are used to derive the weightage across reactions and construct an FDSeM 824. The rows indicate source (pre-cursor) metabolites and columns indicate target metabolites. Interpretability with FDSeM 824 is achieved by ordering the metabolites in FDSeM based on attributes. The ordering is non-trivial and requires a metabolite attribute database and an optimization framework. Embodiments of the present disclosure propose the use of a database of metabolite attributes viz. their identifier (KEGG/MetCyc), SMILES formula, pathway associations, chemical properties (mass, charge, polarity, solubility), which can be used to impose constraints while aligning the FDSeM of organisms.

In some embodiments, the metabolites can be grouped according to constraints. Non-limiting examples of constraints include clustering of metabolites that belong to similar pathways, clustering by optimization of metabolites that have similar chemical properties, and grouping metabolites with similar production profiles by matrix bi-clustering.

A process for comparison of yields for a compound-of-interest may be performed according to the following:
a) Create FDSeM of organisms
b) Use the FDSeM of one organism as a reference and interactively impose a metabolite ordering based on pathway occurrence using the database
c) Utilize a Global Optimization to maximize neighborhood of similar metabolites based on imposed constraint (optimization 1)
d) Perform a bi-clustering on the FDSeM of the reference organism to generate a ‘metabolite fingerprint’
e) Perform a referenced bi-clustering to align the second organism's metabolites globally (pathway level) and locally (metabolite id level) (optimization 2)
f) Sum column values of weighted flux allows to derive the scaled production of compounds in the organisms

A metabolite database can be used to impose a constraint on grouping metabolites based on chemical similarity before optimization 1. Post alignment with organism 2, the production profiles of all metabolites with chemical similarity to compound-of-interest can be assessed quantitatively. A decision on organism choice can be made based on: (1) maximum native yields of compound-of-interest and/or (2) minimum amount of contaminating metabolites present in each organism.

It may also be useful to compare two competing organisms for extraction of a compound. Once the natural yield of a compound-of-interest has been quantitated in each organism, efforts may be directed towards increasing the yield. One way to achieve this is by deletion or overexpression of enzymes that may allow more product to be shunted towards making the compound-of-interest. The deletion/overexpression strategies can be constrained interactively, and simulated. The problem arises when some modifications may benefit the yield in silico, but will be detrimental to growth and survival of the organism. This presents a complex choice and cannot be determined without a simulation and analysis platform, as described herein.

In accordance with at least some embodiments, interpretability is possible by using the RiG 928 to decide the right genetic modifications to optimize yield. An example, but non-limiting, process is proposed to help decide the right genetic modifications to optimize yield:
a) Construction of a RiG 828 post-simulation (FBA/FVA) for organism/organisms being compared.
b) User 208 assesses RiG 828 and performs the knock-out or overexpression of gene (through gene-protein rules database) in the simulation software package 204.
c) Representation interface then globally groups survival reactions with toxicity associated reactions of Organism A by fetching reaction attributes from database.
d) The RiG of both organisms are aligned by:
   (i) Bi-clustering the reference organism RiG using a weighted flux; and/or
   (ii) Aligning the RiG of Organism B with A using optimization
e) An aligned and updated RiG is created for both organisms (alignment method may be by optimization and bi-clustering).
f) A reaction attribute database may be used to identify reactions associated with survival, growth (clubbed as biomass objective function), and toxicity for both organisms.
g) If a gene modification affects survival and growth reactions or promotes toxicity reactions, it is excluded and a new iteration is initiated.
h) Results of gene modifications are compared using FDSeM productivity graphs. Those which enhance the compound-of-interest while promoting flux for biomass objective function are stored for decision making and ranked in order of maximal to minimal effect.
i) A user 208 can access gene modifications for organism(s) that enhanced a compound of interest without affecting survival.

If there is a major contaminant with a similar chemical profile as compound-of-interest, then the user 208 can use the simulations with genetic modification and RiG to mine out favorable and minimal gene deletions that will ensure better yield of compound-of-interest, minus impurities. According to at least some embodiments, the process for minimizing contaminants and ensuring ease of extraction for a compound-of-interest may include:
a) Fetching reactions associated with the contaminant using a database.
b) Interactively deleting the genes associated with the reactions (e.g., using a gene-protein association database).
c) Computing the RiG for each deletion and perform iterative deletions to:
   (i) minimize yield of contaminant;
   (ii) optimize yield of compound-or-interest;
   (iii) optimize the biomass objective function; and/or
   (iv) minimize toxicity.

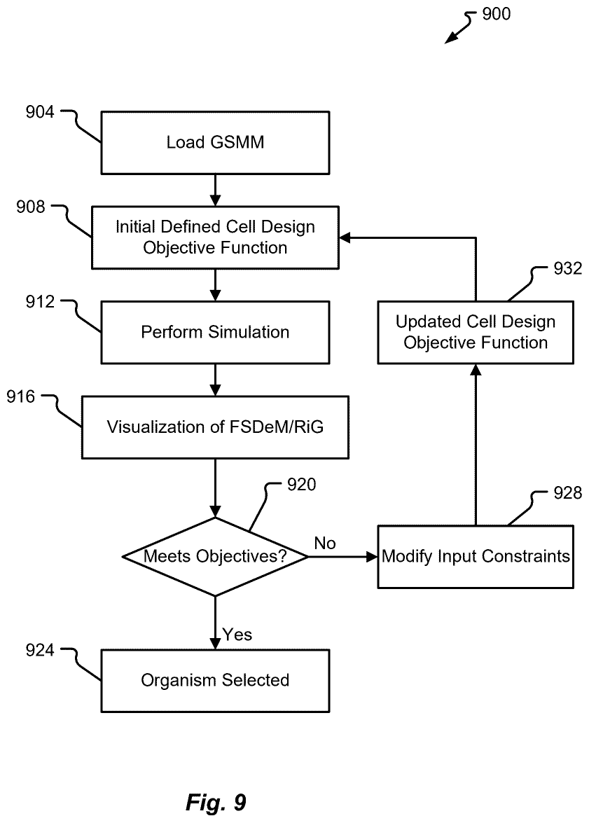

Referring now to FIG. 9, additional details of a process 900 for selecting an organism will be described in accordance with at least some embodiments of the present disclosure. The process 900 begins by loading a GSMM into the simulation software package 204 (step 904). In some embodiments, the GSMM may include one or more reaction pathways comprising one or more intermediate metabolites required to produce the target substance. The GSMM may also include an association between a first reaction and a second reaction present in reaction pathways and a plurality of genes, where the plurality of genes are used to produce the target substance.

The process 900 continues with the simulation software package 204 initially defining a cell design objective function (step 908). The cell design objective function may be defined, at least in part, based on user 208 inputs provided to the simulation software package 204. The initiate cell design objective function may provide an indication of a target compound production amount.

The process 900 may further continue by performing a simulation 912 with the GSMM and the objective function (step 912). In some embodiments, the simulation software package 204 may be configured to make the simulation data (e.g., inputs and/or outputs) interpretable to the user 208 based on the objective function (step 916). Embodiments of the present disclosure contemplate multiple mechanisms for making the simulation data interpretable through the visualization. One mechanism is the FDSeM and the other mechanism is the RiG. As discussed herein, weighted fluxes due to uptake of nutrients in media is represented in a source-target metabolite matrix. The internal fluxes can be computed for each reaction in the organism and the FDSeM allows to assess net depletion of metabolites and net production of metabolites. The net production of metabolites can be analyzed by summing the columns of the FDSeM. The RiG, on the other hand, may be constructed from weighted fluxes from source-target reactions present in a GSMM. A link exists between a source-to-target reaction when the products of source reaction become inputs of target reaction. Hence this network topology captures the reaction flow of the organism. Further the weighted flux populating the RiG provide an estimate of high flow and low flow paths that may lead to a product of interest. The RiG is also associated with gene-protein rules database, where genes that control enzymes for a reaction are stored.

The process 900 may further include determining if the simulation performed in step 912 meets the cell design objective functions defined in step 908 (step 920). If the query is answered affirmatively, then the organism may be selected (step 924). On the other hand, if the query of step 920 is answered negatively, then the input constraints may be modified through the simulation software package 204 (step 928). The cell design objective function may also be updated based on the modified input constraints (step 932). The process 900 may then revert back to step 908 or 912 where an iteration of the simulation is performed.

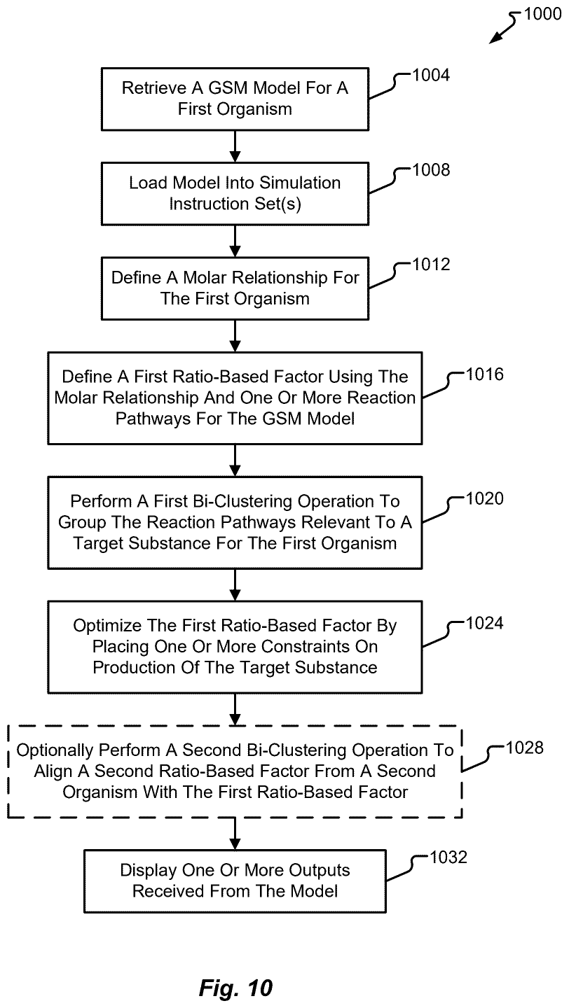

Referring now to FIG. 10, additional details of another process 1000 for simulating and then selecting an organism will be described in accordance with at least some embodiments of the present disclosure. Steps of process 1000 may be combined with steps of any other process depicted and described herein, in any order. For instance, one or more steps of process 1000 may be combined with one or more steps of process 800 and/or process 900 without departing from the scope of the present disclosure.

The process 1000 begins by retrieving a GSMM for a first organism (step 1004). In some embodiments, the GSMM may be loaded from memory 106, from data storage 108, from memory 126, and/or from data storage 128. The GSMM may include a plurality of reaction pathways comprising one or more intermediate metabolites required to produce the target substance.

The process 1000 continues by loading the GSMM into the simulation instruction set(s) 118 (step 1008). A molar relationship may then be defined for the first organism using the simulation software package 204 (step 1012). In some embodiments, the molar relationship includes stoichiometry between the consumed metabolite and the target substance.

The process 1000 may further include defining a first ratio-based factor (step 1016). The first ratio-based factor may be defined using the molar relationship and the reaction pathway(s) for the GSMM. In some embodiments, the first ratio-based factor may establish a relationship between a production rate of the target substance and a consumption rate of the consumed substance.

The process 1000 may further include performing a first bi-clustering operation to group two or more of the reaction pathways associated with the target substance (step 1020) and optimizing the first ratio-based factor by placing one or more constraints on the production of the target substance (step 1024). In some embodiments, the first bi-clustering operation is performed with a constraint that minimizes one or more undesirable substances. In some embodiments, the first bi-clustering operation is performed with an additional constraint to reduce production of one or more substances having a chemical signature that is substantially similar to the one or more undesirable substances. The additional constraint may be applied on at least one of a solubility and a polarity of the one or more undesirable substances. In some embodiments, the first bi-clustering operation is performed with an additional constraint to maintain a ratio of the target substance to the one or more undesirable substances at a value greater than or equal to one.

With respect to the one or more constraints (or the additional constraint), the one or more constraints placed on the production of the target substance include at least one of: (i) defining a maximum amount of the target substance to produce and (ii) defining a minimum amount of the target substance to produce. Alternatively or additionally, the one or more constraints placed on the production of the target substance may include defining an amount of the consumed substance available to use in the production of the target substance.

The process 1000 may further include an optional step of performing a second bi-clustering operation to align a second ratio-based factor from a second organism with the first ratio-based factor (step 1028). It should be appreciated that the first organism and the second organism may both be capable of producing the target substance.

The process 1000 may also include displaying one or more simulation outputs (step 1032). In some embodiments, the simulation output(s) may correspond to outputs generated by the simulation software package 204 that are rendered on a display device. The output(s) may include a number of data and/or graphical outputs generated based on the processing of the GSMM under the defined constraints, as described above.

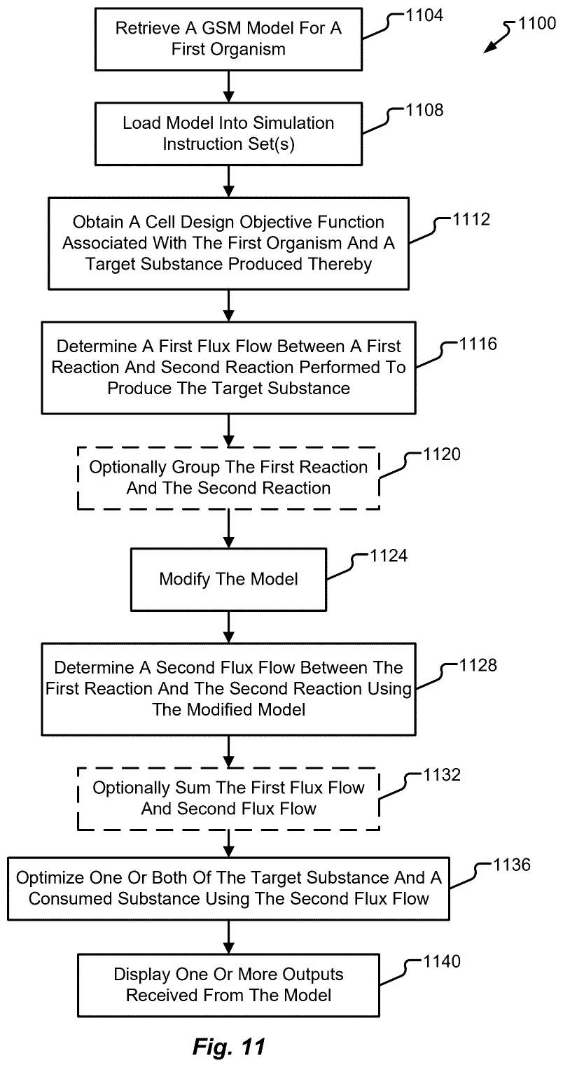

Referring now to FIG. 11, another process 1100 for simulating and then selecting an organism will be described in accordance with at least some embodiments of the present disclosure. Steps of process 1100 may be combined with steps of any other process depicted and described herein, in any order. For instance, one or more steps of process 1100 may be combined with one or more steps of process 800, process 900, and/or process 1000 without departing from the scope of the present disclosure.

The process 1100 begins by retrieving a GSMM from an appropriate data storage location (e.g., memory and/or database) (step 1104). The GSMM may be loaded from a database of models that are curated and indexed by organism type. The GSMM may be associated with a first organism and may express a plurality of reaction pathways comprising one or more intermediate metabolites to produce a target substance from a consumed substance in the first organism. The GSMM may also provide an association between a first reaction and a second reaction present in each reaction pathway and one or more genes that are used to produce the target substance.

The GSMM is then loaded into the simulation instruction set(s) 118 (step 1108). The process 1100 may also include obtaining a cell design objective function associated with the first organism and a target substance produced thereby (step 1112). Specifically, but without limitation, the cell design objective function may include one or more constraints on production of the target substance that are useable to optimize the production of the target substance. In some embodiments, the cell design objective function includes at least one of a minimization function (e.g., minimize unwanted byproducts) and a maximization function (e.g., maximize target substance production). In some embodiments, the one or more constraints include a constraint on at least one of a pathway association, a chemical property, a metabolite attribute, and/or a formula. The one or more constraints may also include defining an amount of the consumed substance available to use in the production of the target substance.

The process 1100 may also include determining a first flux flow between a first reaction and a second reaction (step 1116). The first reaction and the second reaction may both be performed to produce the target substance using the consumed substance.

The process 1100 may include an optional step of grouping the first reaction and the second reaction (step 1120). In some embodiments, the first reaction and second reaction may be grouped using a plurality of survival reactions, where the plurality of survival reactions occur as part of maintaining the first organism. In some embodiments, the first reaction and the second reaction may be grouped using a toxicity measure associated with the one or more intermediate metabolites.

The process 1100 may further include modifying the GSMM (step 1124). Modifying the GSMM may include changing at least one of the association between the first reaction and the second reaction to yield a modified association and by changing the one or more genes that are used to produce the target substance. Such an approach may yield a modified GSMM.

The process 1100 may further include determining a second flux flow between the first reaction and the second reaction using the modified GSMM (step 1128). An optional step in process 1100 may further include summing the first flux flow and the second flux flow as part of obtaining a net production of metabolites (step 1132). The flux flow can be converted to production by normalizing the concentration in mmoles. Production may be calibrated with respect to input substrate supplied per unit time.

The process 1100 may also include optimizing one or both of the target substance and the consumed substance using the second flux flow (step 1136). As discussed herein, the optimization may include efforts to maximize and/or minimize various outputs of the reaction(s). Such optimizations may be defined with the help of user 208 input provided to the simulation software package 204. Accordingly, the process 1100 may further include displaying one or more outputs of the simulation on a display device (step 1140). The output(s) displayed as part of the simulation may enable the user 208 to decide on defining one or more inputs and/or one or more reaction to use as part of producing a target substance using the one or more inputs.

In the foregoing description, for the purposes of illustration, methods were described in a particular order. It should be appreciated that in alternate embodiments, the methods may be performed in a different order than that described without departing from the scope of the embodiments. It should also be appreciated that the methods described above may be performed as algorithms executed by hardware components (e.g., circuitry) purpose-built to carry out one or more algorithms or portions thereof described herein. In another embodiment, the hardware component may comprise a general-purpose microprocessor (e.g., CPU, GPU) that is first converted to a special-purpose microprocessor. The special-purpose microprocessor then having had loaded therein encoded signals causing the, now special-purpose, microprocessor to maintain machine-readable instructions to enable the microprocessor to read and execute the machine-readable set of instructions derived from the algorithms and/or other instructions described herein. The machine-readable instructions utilized to execute the algorithm(s), or portions thereof, are not unlimited but utilize a finite set of instructions known to the microprocessor. The machine-readable instructions may be encoded in the microprocessor as signals or values in signal-producing components by, in one or more embodiments, voltages in memory circuits, configuration of switching circuits, and/or by selective use of particular logic gate circuits. Additionally or alternatively, the machine-readable instructions may be accessible to the microprocessor and encoded in a media or device as magnetic fields, voltage values, charge values, reflective/non-reflective portions, and/or physical indicia.

The present invention, in various embodiments, configurations, and aspects, includes components, methods, processes, systems and/or apparatus substantially as depicted and described herein, including various embodiments, subcombinations, and subsets thereof. Those of skill in the art will understand how to make and use the present invention after understanding the present disclosure. The present invention, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and\or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments, configurations, or aspects for the purpose of streamlining the disclosure. The features of the embodiments, configurations, or aspects of the invention may be combined in alternate embodiments, configurations, or aspects other than those discussed above. This method of disclosure is not to be interpreted as reflecting an intention that the claimed invention requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the invention.

Moreover, though the description of the invention has included description of one or more embodiments, configurations, or aspects and certain variations and modifications, other variations, combinations, and modifications are within the scope of the invention, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

What is claimed is:

1. A method, comprising:
loading a model associated with a first organism, wherein the model expresses a plurality of reaction pathways comprising one or more intermediate metabolites to produce a target substance from a consumed substance in the first organism, and wherein the model further provides an association between a first reaction and a second reaction present in each reaction pathway and one or more genes that are used to produce the target substance;
obtaining a cell design objective function that includes one or more constraints on production of the target substance that are useable to optimize the production of the target substance;
determining a first flux flow between the first reaction and the second reaction, wherein the first reaction and the second reaction are performed to produce the target substance using the consumed substance;
modifying the model by changing at least one of the association between the first reaction and the second reaction to yield a modified association and by changing the one or more genes that are used to produce the target substance, thereby providing a modified model;
determining a second flux flow between the first reaction and the second reaction using the modified model; and
optimizing one or both of the target substance and the consumed substance using the second flux flow.

2. The method of claim 1, further comprising:
grouping the first reaction and the second reaction using a plurality of survival reactions, wherein the plurality of survival reactions occur as part of maintaining the first organism.

3. The method of claim 1, further comprising:
grouping the first reaction and the second reaction using a toxicity measure associated with the one or more intermediate metabolites.

4. The method of claim 1, wherein the model comprises a genomic-scale metabolic (GSM) model.

5. The method of claim 1, wherein the cell design objective function comprises at least one of a minimization function and a maximization function.

6. The method of claim 1, further comprising:
summing the first flux flow and the second flux flow as part of obtaining a net production of metabolites.

7. The method of claim 1, wherein the model is loaded from a database of models that are curated and indexed by organism type.

8. The method of claim 1, wherein the one or more constraints comprises a constraint on at least one of a pathway association, a chemical property, a metabolite attribute, and a formula.

9. The method of claim 1, wherein the one or more constraints placed on the production of the target substance includes defining an amount of the consumed substance available to use in the production of the target substance.

10. A system, comprising:
a processor; and
a memory device coupled with the processor, wherein the memory device comprises data stored thereon that, when processed by the processor, enables the processor to:
load a model associated with a first organism, wherein the model expresses a plurality of reaction pathways comprising one or more intermediate metabolites to produce a target substance from a consumed substance in the first organism, and wherein the model further provides an association between a first reaction and a second reaction present in each reaction pathway and one or more genes that are used to produce the target substance;
obtain a cell design objective function that includes one or more constraints on production of the target substance that are useable to optimize the production of the target substance;
determine a first flux flow between the first reaction and the second reaction, wherein the first reaction and the second reaction are performed to produce the target substance using the consumed substance;
modify the model by changing at least one of the association between the first reaction and the second reaction to yield a modified association and by changing the one or more genes that are used to produce the target substance, thereby providing a modified model;
determine a second flux flow between the first reaction and the second reaction using the modified model; and
optimize one or both of the target substance and the consumed substance using the second flux flow.

11. The system of claim 10, wherein the data further enables the processor to group the first reaction and the second reaction using a plurality of survival reactions, wherein the plurality of survival reactions occur as part of maintaining the first organism.

12. The system of claim 10, wherein the data further enables the processor to group the first reaction and the second reaction using a toxicity measure associated with the one or more intermediate metabolites.

13. The system of claim 10, wherein the model comprises a genomic-scale metabolic (GSM) model.

14. The system of claim 10, wherein the cell design objective function comprises at least one of a minimization function and a maximization function.

15. The system of claim 10, wherein the data further enables the processor to sum the first flux flow and the second flux flow as part of obtaining a net production of metabolites.

16. The system of claim 15, wherein the model is loaded from a database of models that are curated and indexed by organism type.

17. The system of claim 16, wherein the one or more constraints comprises a constraint on at least one of a pathway association, a chemical property, a metabolite attribute, and a formula.

18. The system of claim 15, wherein the one or more constraints placed on the production of the target substance includes defining an amount of the consumed substance available to use in the production of the target substance.

19. The system of claim 10, further comprising:
a user interface that enables a display of one or more outputs received from the model and that enables a user to define the one or more constraints for a simulation.

20. A non-transitory computer-readable medium comprising processor-executable instructions stored thereon, wherein the instructions enable a processor, when executed, to:
load a model associated with a first organism, wherein the model expresses a plurality of reaction pathways comprising one or more intermediate metabolites to produce a target substance from a consumed substance in the first organism, and wherein the model further provides an association between a first reaction and a second reaction present in each reaction pathway and one or more genes that are used to produce the target substance;
obtain a cell design objective function that includes one or more constraints on production of the target substance that are useable to optimize the production of the target substance;
determine a first flux flow between the first reaction and the second reaction, wherein the first reaction and the second reaction are performed to produce the target substance using the consumed substance;
modify the model by changing at least one of the association between the first reaction and the second reaction to yield a modified association and by changing the one or more genes that are used to produce the target substance, thereby providing a modified model;
determine a second flux flow between the first reaction and the second reaction using the modified model; and
optimize one or both of the target substance and the consumed substance using the second flux flow.
